(19) United States
(12) Patent Application Publication
(10) Pub. No.: US 2009/0168068 A1
(43) Pub. Date: Jul. 2, 2009

(54) Coated metal surface on solid support for displacement reactions

(75) Inventors: Per Mansson, Sollentuna (SE); Bo Liedberg, Linkoping (SE)

Correspondence Address: BACON & THOMAS, PLLC, 625 SLATERS LANE, FOURTH FLOOR, ALEXANDRIA, VA, 22314-1176, US

(73) Assignee: BIOSENSOR APPLICATION SWEDEN AB, Sundbyberg (SE)

(21) Appl. No.: 12/289,218

(22) Filed: Oct. 23, 2008

Related U.S. Application Data
(63) Continuation of application No. 10/517,320, filed on Jul. 21, 2005, now abandoned.
(60) Provisional application No. 60/389,497, filed on Jun. 19, 2002.

(30) Foreign Application Priority Data
Jun. 19, 2002 (SE) 0201874-5

(51) Int. Cl. G01N 21/55; G01N 33/553; B05D 1/36; B32B 15/04

(52) U.S. Cl. 356/445; 436/525; 427/414; 428/457; 527/200

(57) ABSTRACT

A coated metal surface on a solid support, wherein the coating consists of a self-assembled monolayer (SAM) of oligo(ethylene glycol)-terminated amide group-containing alkyl thiols firmly attached to the metal surface via the thiol-end and low molecular weight antigens bound via an amide-group to the SAM-forming OEG molecule, wherein the alkyl portion has 1-20 methylene groups, wherein the oligo(ethylene glycol) portion has 1-15 ethylene oxy units, and wherein the antigens, such as explosives and narcotics, are optionally reversibly bound to antibodies specific for the antigens, is disclosed. The coated metal surface on a solid support may be used in a method of detecting analyte antigens as part of an analysis device, such as a Piezoelectric Crystal Microbalance device or a Surface Plasmon Resonance biosensor, for detection in an aqueous solution of an analyte antigen with higher affinity to an antibody than the antigen of the coating by monitoring the displacement of the antibody from the coating.

The present invention relates to a coated metal surface on solid support for displacement reactions, especially for analyte detection in an aqueous solution by displacement from the metal surface coating of reversibly bound antibodies specific for the analyte. Detection of the displacement, and thus the presence of the analyte in an aqueous solution is performed with a analysis device, such as a Piezoelectric Crystal Microbalance (PCM) device or a Surface Plasmon Resonance (SPR) biosensor.

### BACKGROUND

The SPR biosensor is a sensitive real-time technique, which can be used to extract information about molecular interaction near certain metal surfaces. It offers the possibility to determine concentration, association and dissociation rate constants and affinity as well as epitope mapping and determination of interaction specificity [B. Liedberg and K. Johansen, Affinity biosensing based on surface plasmon detection in “Methods in Biotechnology, Vol. 7: Affinity Biosensors: Techniques and Protocols”, K. R. Rogers and A. Muchandani (Eds.), Humana Press Inc., Totowa, N.J., pp. 31-53]. One of the components participating in the studied reaction is immobilized on the metal surface either before or during the SPR experiment. The immobilized molecule is exposed to a continuous flow into which one can inject interacting species. The method is based on optical detection and the sensing signal reflects changes in dielectric function or refractive index at the surface. These changes can be caused by molecular interaction at the surface.

The PCM technique is based on an oscillating piezoelectric crystal in a microbalance device, wherein the crystal consists of e.g. quartz, aluminum nitride (AlN) or sodium potassium niobiates (NKN). When the crystal is a quarts crystal, the device is referred to as a QCM (quartz crystal microbalance). The PCM and QCM are gravimetrical sensors and are thus sensitive to mass changes. A QCM comprises a piezoelectric quartz crystal plate upon which metal electrodes have been deposited on both sides. An alternating potential difference applied on such a crystal plate induces shear waves. At certain frequencies—such that the thickness is an odd integer of half wavelengths—the crystal will be in resonance [M. Rodahl, F. Höök, A. Krozer, P. Brzezinski and B. Kasemo, Quartz crystal microbalance setup for frequency and Q-factor measurements in gaseous and liquid environments, Review of Scientific Instruments 66 (1995) pp. 3924-3930]. The energetically most favourable number of half wavelengths is one. The resonance frequency is dependent on the thickness of the crystal, but is normally in the MHz range. A mass change on the surface of the plate will result in a shift in the resonance frequency. The fact that frequency shifts of 0.01 Hz can be easily measured makes the QCM a sensitive sensor for determining mass variations. A number of patents and other publications describe the use of piezoelectric quartz crystals (QCM) as affinity-based chemical sensors/detectors in e.g. various immunoassay techniques, and detection of bacteria and virus. In most of these applications the QCM-instrument is used to analyze the weight gain of the crystal after interaction between interaction pairs, e.g. antibodies and antigens.

There are obvious difficulties in analyzing small molecules with conventional immunosensors due to the low response, i.e. small change in weight of the sensor crystal. For attaining the necessary detection of small molecules, the sensitivity of the system has to be improved. To improve the detectability of small molecules, they should be reacted with larger molecules by specific interaction between the small molecules and the larger ones. For example, small antigen molecules are reacted with antibodies specifically binding to them to form antigen-antibody complexes that are easier to detect. If an antigen derivative with less affinity to the antibody than the analyte antigen is immobilized to a surface, antibodies specific for these antigens may be reversibly bound to the immobilized antigen. Then, when the analyte antigen is present in a solution, the antibody will be displaced from the immobilized antigen and form an antigen-antibody complex with the analyte antigen. In case the antibody carries a marker, such as a fluorescent label, the formed complex can be detected with the aid of the marker. On the other hand, if the immobilized antigen is immobilized on a surface of a biosensor sensitive for mass changes, then the displacement of the antibody from the surface will result in a weight loss. Such displacement reactions are used in the present invention.

An organosulphur compound, such as an alkyl thiol, can be used to form a well-ordered and densely packed SAM on a gold substrate. The strong chemical bond between sulphur and gold couples the molecules to the surface. Once pinned to the substrate, which occurs within seconds, the molecules start to organize themselves into densely packed formations due to the van der Waal forces between the alkyl chains. The latter process is time consuming and it takes hours or even days before a well-ordered SAM is completed. The length of the molecules used has a strong influence on the properties of the obtained SAM. An all trans SAM is a perfectly ordered and densely packed monolayer, whereas a SAM of poorer quality possesses defects like complete or terminal gauche (more or less spaghetti-like). The molecules in a SAM will display a chain tilt of 25-40° due to the mismatch between the pinning distance and size of the van der Waal diameter of the carbon chains [B. Liedberg and J. M. Cooper, Bioanalytical applications of self-assembled monolayers in “Immobilized Biomolecules in Analysis: A Practical Approach”, T. Cass and F. S. Liegler (Eds.), Biosensors, Oxford university press, Oxford, pp. 55-78]. The free end of the molecule can be linked to desired groups or even proteins. In this way it is possible to design surfaces with interesting and useful properties. Mixing different alkyl thiols further increases the versatility. However, it should be noted that a certain mixture of thiols in a loading solution does not necessarily result in a SAM of the same mixture. On the contrary, this is seldom the case due to complex thermodynamic processes taking place during the self-assembly. To our knowledge, SAM coupled to a low molecular weight antigen has not been developed for displacement reactions where an antigen-specific antibody is reversibly bound to the immobilized antigen and dissociates in aqueous solution and binds to an analyte antigen that has a higher affinity to the antibody than the immobilized antigen.

### DESCRIPTION OF THE INVENTION

The present invention provides a coated metal surface on a solid support that is useful in an analysis device for detection of an analyte antigen in an aqueous solution by monitoring displacement of an antibody reversibly bound to an antigen on the coating by dissociation and reaction with the analyte antigen.

In this specification and claims the word antibody is intended to comprise whole antibodies or antigen-binding parts of antibodies or synthetic antigen-binding molecules.

Thus, one aspect of the invention is directed to a coated metal surface on a solid support, wherein the coating consists of a self-assembled monolayer (SAM) of oligo(ethylene glycol)-terminated amide group-containing alkyl (OEG) thiols. The OEG thiols contain a SH group that is firmly attached to the metal surface and a low molecular weight antigen introduced via amide-groups to the SAM-forming OEG thiol molecule, wherein the alkyl portion has 1 to 20 methylenes, the OEG portion has 1 to 15 ethylene oxy units, and wherein the antigens are optionally reversibly bound to antibodies specific for the antigens.

In an embodiment of the invention, the oligo(ethylene glycol) has 4-6 ethylene oxy units and the alkyl group has 15 methylene units.

The low molecular weight antigens are synthetically bound to the OEG molecules prior to SAM formation by reacting functional groups on the antigens with functional groups terminating the OEG thiol. These functional groups can be of the type-carboxylic acid, amino and hydroxyl groups. It may be necessary to introduce a functional group on the low molecular weight antigen prior to the reaction in case the antigen lacks functional groups for the reaction.

The coated metal surface on a solid support according to the invention will usually be stored separately from the antigen-specific antibodies prior to use. When used in displacement analysis, the coated metal surface on a solid support will, however, comprise the specific antibodies reversibly bound to the antigens of the coating.

The metal of the coated metal surface on a solid support according to the invention is preferably selected from e.g. the group consisting of gold, silver, aluminum, chromium and titanium. The presently preferred metal is gold.

The antigen of the coating is the same as or a derivative of the analyte antigen except that it is immobilized through a bond to the SAM. The antigen of the coating may thus be derivatized to optimize dissociation of the bound antibody in an aqueous solution.

The antigens bound to the SAM of the coating according to the invention are the same or different, i.e. the antigens may bind to the same specific antibodies or there may be a mixture of two or more bound antigens binding to different specific antibodies enabling the detection of the presence of several different analyte antigens in an aqueous solution. In case the antibodies carry different markers, such as fluorescent markers, it will be possible to detect displacement of the different antibodies. However, a mixture of several different antibodies will normally be used in cases where screening of samples for any of the target antigens is sufficient, such as screening of samples for any narcotics or explosives. In order to avoid interferences between the different antigens and antibodies, having different affinities to each other, it may be necessary to introduce discrete patches or microarrays of spots of coatings with the different antigens on the solid support.

In a preferred embodiment of the invention the antigen of the coating is selected from the group consisting of optionally derivatized explosives and narcotics. In case the selected antigen of the coating binds too strongly to the specific antibody so that the dissociation of the antibody in aqueous solution is hampered, the antigen molecule may be chemically modified, e.g. by modification of functional groups such as ester or amino groups (by removal of, or replacing the original groups) or by eliminating a part of the antigen molecule, or introducing new functional groups or side chains to the antigen molecule, to reduce its affinity to the antibody.

The explosives are preferably selected from the group consisting of trinitrotoluene (TNT), dinitrotoluene (DNT), hexahydro-1,3,5-trinitro-1,3,5-triazine (RDX), octahydro-1,3,5,7-tetranitro-1,3,5,7-tetrazine (HMX), pentaerythritol tetranitrate (PETN), and nitroglycerine (NG), and the narcotics are preferably selected from the group consisting of cocaine, heroine, amphetamine, methamphetamine, cannabinols, tetrahydrocannabinols (THC), and methylenedioxy-N-methylamphetamine (Ecstacy).

In a presently preferred embodiment the solid support of the coated metal surface on a solid support according to the invention is a piezoelectric quarts crystal electrode or a glass plate or prism, and thus the coated metal surface on the piezoelectric crystal electrode is suitable for use in a PCM device and the coated metal surface on a glass plate or prism is suitable for use in a SPR apparatus.

Another aspect of the invention is directed to the use of the coated metal surface on a solid support according to the invention as part of an analysis device for detection in an aqueous solution of analyte antigens with higher affinity to specific antibodies than the antigens of the coating by monitoring the displacement of the antibodies from the coating.

Yet another aspect of the invention is directed to a method of detecting analyte antigens in an aqueous solution comprising activating, if necessary, the coated metal surface on a solid support according to the invention lacking bound antibodies by bringing antigen-specific antibodies into contact with the coated metal surface in an aqueous solution, allowing binding of the antibodies to the antigens of the coating, removing excess antibodies, bringing the aqueous solution possibly containing the analyte antigens that have higher affinity to the antibodies than the antigens of the coating into contact with the antibodies reversibly bound to the coating, allowing the antibodies to dissociate and react with the analyte antigens, and detecting the loss of mass on the coated metal surface by means of an analysis device.

In an embodiment of the method of the invention the analysis device is selected from the group consisting of a Piezoelectric Quarts Crystal Microbalance device and a Surface Plasmon Resonance biosensor.

In a presently preferred embodiment the analysis device comprises a flow cell in which the coated metal surface on a solid support according to the invention is placed.
The invention will now be illustrated by some drawings and description of experiments, but it should be understood that the invention is not intended to be limited to the specifically described details.

### DESCRIPTION OF THE DRAWINGS

FIG. 1 shows the chemical formula of some narcotics, Cocaine, Heroine, Amphetamine, Ecstacy, Methamphetamine, Cannabinol and Tetrahydrocannabinol (THC).

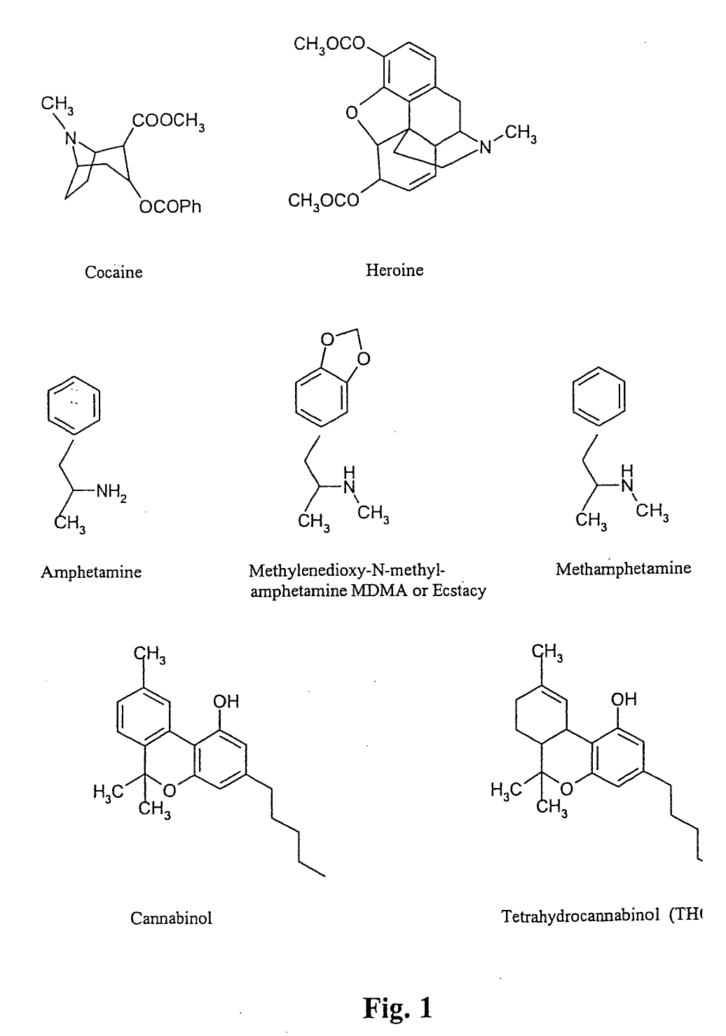

FIG. 2 shows the chemical formula of trinitrotoluene (TNT) and 2,4-dinitrotoluene (2,4-DNT).

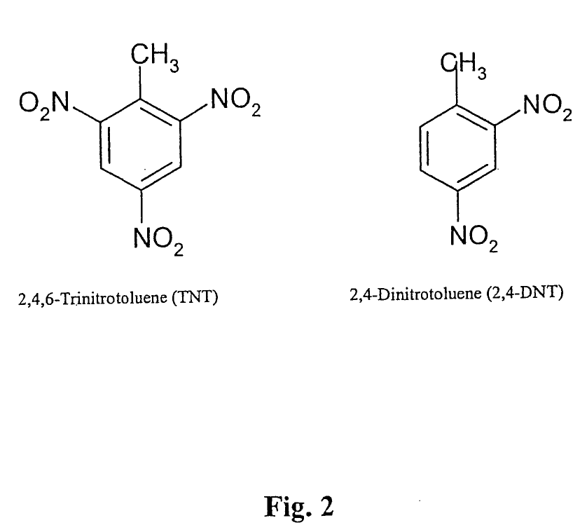

FIG. 3 shows schematically the displacement mechanism taking place on the metal surface of a solid support, e.g. a QCM electrode. Note that the representation is not to scale. In reality an antibody is much larger than a TNT molecule. The sensor surface displayed is based on the self-assembly (SAM) technique. The derivatized TNT molecules, TNT-analogs, are covalently bound to the metal surface via the SAM, the ABTNT antibodies specific for the TNT and TNT-analog is at first reversibly, loosely, bound to the TNT-analogs, and at exposure to TNT in solution, the ABTNT dissociates and forms a complex with TNT.

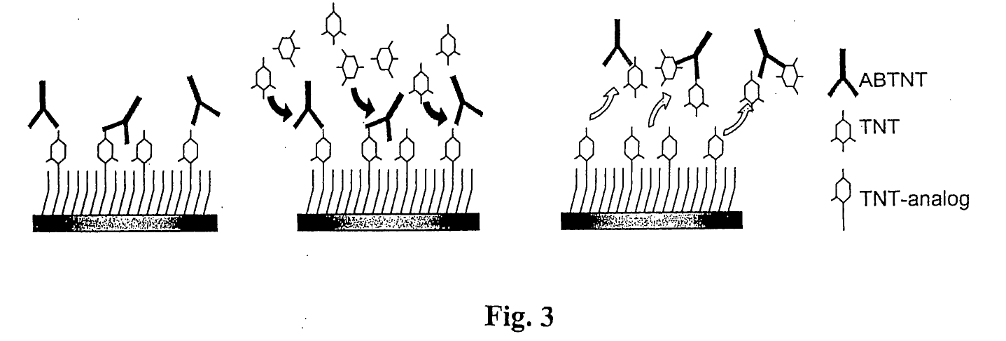

FIG. 4 shows the chemical structures of EG₄ and EG₆.

FIG. 5 shows the chemical structure of ANA1, ANA2 and ANA3.

FIG. 6 shows adsorption of antibodies, after 30 min incubation in ABTNT (0.02 g/L), to different TNT-analogues at different mixing ratios with EG₄. An excellent agreement is observed between the two techniques, IRAS and null ellipsometry.

FIG. 7 shows the ABTNT-binding capacity observed for EG₄ and ANA1, and mixtures thereof, by the real-time technique SPR (Biacore2000). The flow rate was set to 50 μL/min and a volume of 100 μL of ABTNT (0.02 g/L) was injected. Note the low adsorption onto the SAM of EG₄.

FIG. 8 shows ABTNT-binding capacity observed for EG₄ and ANA1, and mixtures thereof, by the real-time technique QCM (V2B). The flow rate was set to 50 μL/min and a volume of 100 μL of ABTNT (0.02 g/L) was injected. Note the low adsorption onto the SAM of EG₄.

FIG. 9 shows experiments on a Biacore2000 instrument showing SPR response to TNT injections of 1, 10 and 100 pg/μL for SAMs of EG₄ and ANA1, and mixtures thereof. The surfaces had previously been loaded by injections of 100 μL ABTNT. The flow rate was set to 50 μL/min.

FIG. 10 shows QCM measurements performed on a modified V2B flow cell system. The flow was set to 50 μL/min and all TNT injection volumes were 100 μL. The surfaces had previously been loaded with ABTNT (0.02 g/L) by an injection of 100 μL. The SAMs were assembled from loading solutions containing 100% and 50% ANA1. The concentrations of TNT in the injections were 1, 10 and 100 pg/μL and they were made in series, leaving the surface previously exposed to TNT for the second and third TNT injection. The arrows show the injections.

FIG. 11 shows QCM measurements performed on a modified V2B flow cell system. The flow was set to 50 μL/min and all TNT injection volumes were 100 μL. The surfaces had previously been loaded with ABTNT (0.02 g/L) by an injection of 100 μL. The SAMs were assembled from loading solutions containing 10% and 1% ANA1. The concentrations of TNT in the injections were 1, 10 and 100 pg/μL and they were made in series, leaving the surface previously exposed to TNT for the second and third TNT injection. The arrows show the injections.

### DESCRIPTION OF EXPERIMENTS

A mixed monolayer was produced that contained two kinds of molecules, the first being protein repellent and the second being a TNT-analogue, thereby making it possible to obtain SAMs containing a varying amount of analogue that displays low levels of non-specific binding.

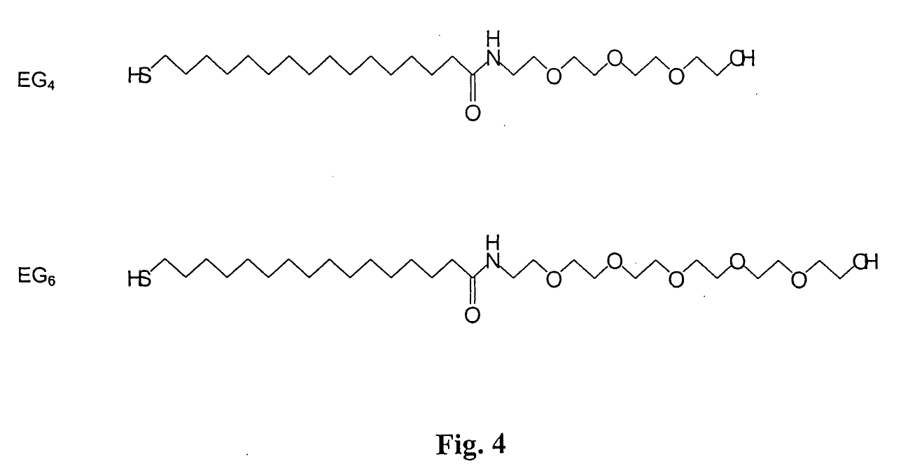

The initial step was to evaluate the protein resistant properties of a SAM constituted of oligo(ethylene glycol) (OEG)-terminated amide group-containing alkyl thiols. The two molecules chosen for this purpose were EG₄ and EG₆ (FIG. 4). Previous reports have shown repellent qualities for these molecules [P. Harder, M. Grunze, R. Dahint, G. M. Whitesides and P. E. Laibinis, Molecular conformation in oligo(ethylene glycol)-terminated self-assembled monolayers on gold and silver surfaces determines their ability to resist protein adsorption, Journal of Physical Chemistry B, 102 (1998) pp. 426-436]. SAMs of pure EG₄ and EG₆, respectively, as well as different compositions of the two were characterized by use of several techniques, namely null ellipsometry, contact angle goniometry and infrared reflection absorption spectroscopy (IRAS).

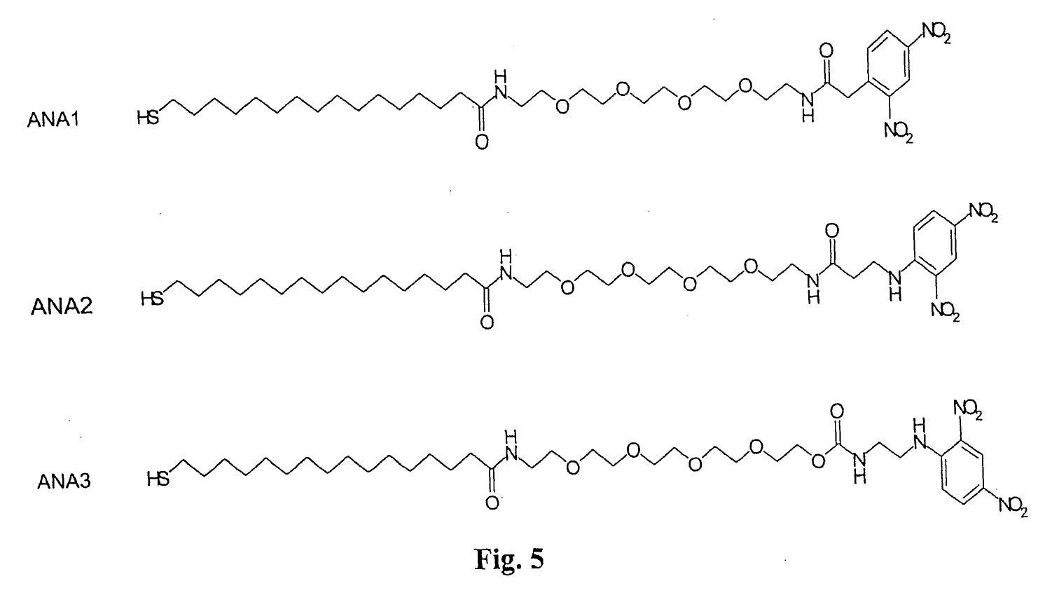

Furthermore, three TNT-analogue molecules (ANA1, ANA2 and ANA3) (FIG. 5), all containing a 2,4-dinitrobenzene end group, were examined separately and in different mixings with a suitable candidate among the OEG compositions mentioned above. These mixed SAMs were then characterized by the same techniques used for the pure OEG SAMs. In addition, SAMs made from 100% analogue—1,2 and 3 separately—were examined with X-ray photoelectron spectroscopy (XPS).

Using null ellipsometry and IRAS, the capacity to bind ABTNT was also determined for all the TNT-analogues at the different mixing ratios. Moreover, the magnitude of displacement of ABTNT in response to exposure to TNT was examined by means of SPR and QCM.

### Compounds

The EG₄ and EG₆ molecules were obtained from the divisions of Applied Physics and Chemistry, Department of Physics and Measurement Technology, Linköping University, Sweden and the analogues ANA1, ANA2 and ANA3 were synthesized at the division of Chemistry, Department of Physics and Measurement Technology, Linköping University, Sweden. ABTNT and TNT were from Biosensor Applications Sweden AB.

### Sample Preparation

Silicon wafers were cleaned in TL2 (MilliQ water:25% hydrogen peroxide:37% hydrogen chloride 6:1:1 at 85° C. for 10 min) and rinsed thoroughly in MilliQ water and dried in nitrogen gas prior to coating of 25 Å of titanium and 2000 Å of gold by electron beam evaporation. The equipment used was a Balzers UMS 500 P system. The evaporation rate was 1 Å/s and 10 Å/s for titanium and gold, respectively. A base pressure of at least 10⁻⁹ was kept and during evaporation the pressure was noted to be on the low 10⁻⁷ scale at all times. This type of surfaces was used for all experiments except SPR and QCM measurements. The SPR surfaces (plain gold) were obtained from Biacore AB, Uppsala, Sweden and the gold-coated QCM crystals were obtained from Biosensor Applications Sweden. It should be noted that the surfaces used for SPR experiments had a similar surface roughness compared to the ones used for the rest of the experiments, whereas the surface coating of the QCM crystals were of a much rougher nature.

Before exposure to thiol loading solutions the sample surfaces were cleaned in TL1 (MilliQ water:25% hydrogen peroxide:30% ammomia 5:1:1 at 85° C. for 10 min) and rinsed thoroughly in MilliQ water. The concentration of thiols in the 99.5% ethanol based loading solutions was 20 μM for pure thiol solutions as well as for mixed thiol solutions. Incubation of the surfaces occurred during approximately 40 h at room conditions. The samples were rinsed in 99.5% ethanol twice and then ultrasonicated for 3 min (since the gold coating on the QCM crystals did not withstand this step, it was omitted) and rinsed two more times in 99.5% ethanol. If not stated otherwise, the surfaces were stored in pure 99.5% ethanol for a maximum of 8 h before they were dried in nitrogen gas and analysed. A number of samples examined with IRAS and null ellipsometry were also subsequently incubated at room conditions for 30 min in ABTNT at a concentration of 0.02 g/L, prepared in PBS (pH7.4) and examined again. At all time the samples were handled with TL1-cleaned forceps.

### Surface Plasmon Resonance (SPR)

For SPR experiments two different types of instruments from Biacore AB, equipped with temperature controlled flow cells, were employed. A series of experiments were performed on a BiacoreX apparatus with two flow channels. For these experiments the flow rate was set to 10 μL/min and the sample surfaces were loaded by a 70 μL injection of ABTNT (0.02 g/L). Subsequently, TNT solutions of concentrations 100 pg/μL and 10 ng/μL were injected in separate flow channels to cause displacement and hence a dissociation of ABTNT from the surface.

The second instrument used was a Biacore2000 equipped with four flow channels. The flow rate was 50 μL/min and all injection volumes were 100 μL. The four flow channels were run in series at all times. Like before, the injected ABTNT had the concentration 0.02 g/L. The concentrations of the TNT injections were 1, 10 and 100 pg/μL.

In all cases the running buffer was PBS (pH7.4) and both ABTNT and TNT solutions were prepared in the same medium. The sample surfaces used for SPR experiment were glass plates coated with about 400 Å gold and the flow cell temperature was kept at 25° C.

### Quartz Crystal Microbalance (QCM)

The QCM measurements were performed at room conditions on a slightly modified flow cell system V2B from Biosensor Applications Sweden AB. The AT-cut QCM crystals used were a thickness shear mode type with a resonance frequency of 10 MHz. The thickness of the deposited titanium and gold layer were 250-300 Å and 400-450 Å, respectively. All parameters were set as in the experiments with Biacore2000, i.e. flow rate 50 μL/min, injection volume 100 μL, ABTNT concentration 0.02 g/L, TNT concentrations 1, 10 and 100 pg/μL and running buffer PBS (pH7.4). It should be noted that the TNT injections were made one after another in the same flow channel, which means that only the 1 pg/μL TNT injection was in fact performed on a TNT-non-exposed surface.

### Results

### OEG Molecules

The OEG molecules EG₄ and EG₆ were used to produce SAMs on gold. Besides pure EG₄ and EG₆ SAMs, two mixed SAMs containing both molecules, were prepared and examined. The latter ones were assembled from loading solutions containing 75% and 50% of EG₄ and the rest EG₆. The mixed monolayers were denoted EG₄:EG₆ 3:1 and EG₄:EG₆ 1:1. The SAM characterization of different compositions of EG₄ and EG₆ with null ellipsometry and contact angle goniometry are given in Table 1. The self-assembly process displays good repeatability and the obtained results agree with recent findings [R. Valiokas, M. Östblom, S. Svedhem, S. C. T. Svensson, and B. Liedberg; Temperature-driven phase transitions in oligo(ethylene glycol)-terminated self-assembled monolayers, The Journal of Physical Chemistry B, 104(32) (2000) pp. 7565-7569, REF.]. The small angles obtained in contact angle goniometry measurements display the low hydrophobicity of these SAMs, which is one of the many prerequisites for repellent properties. A slight increase in thickness can be discerned with increasing proportion of EG₆. As a rule of thumb, hydrophobic surfaces attract proteins and cells. Furthermore, the low value of the hysteresis suggests very homogenous surfaces.

TABLE 1
Characteristics of SAMs of EG₄, EG₆ and mixtures
of the two, given with maximum errors.

| Loading | | Contact angle goniometry (°) | |
|---|---|---|---|
| solution | Thickness of SAM (Å) | Advancing θₐ | Receeding θᵣ |
| EG₄ | 35.7 ± 2.2^{a} (33.9^{e}) | 29 ± 3^{c} (30^{e}) | 24 ± 1^{c} (28^{e}) |
| EG₄:EG₆ 3:1 | 34.4 ± 0.5^{b} | 31 ± 1^{d} | 27 ± 1^{d} |
| EG₄:EG₆ 1:1 | 37.0 ± 1.5^{a} | 33 ± 1^{c} | 30 ± 1^{c} |
| EG₆ | 38.4 ± 0.7^{b} (38.9^{e}) | 28 ± 3^{d} (28^{e}) | 23 ± 1^{d} (25^{e}) |

^{a}3 × 5 measurements on three surfaces,
^{b}2 × 5 measurements on two surfaces,
^{c}three measurements on three surfaces
^{d}two measurements on two surfaces,
^{e}REF Valiokas et al..

### TNT-Analogues

Before the TNT-analogues were mixed with EG₄, they were examined separately in SAMs assembled from loading solutions containing pure ANA1, 2 and 3, respectively. The results from ellipsometry and contact angle measurements are summarized in Table 2. As expected, the thickness of the analogues exceeds that of the OEG molecules. The small differences between the three analogues even reflect the difference in length of the molecules, recalling their chemical structure in FIG. 5. The contact angles noted here are generally larger than those seen for the OEG molecules. Also the hysteresis between advancing and receeding angle is larger and reveals a rougher surface. This would be expected, considering the relatively bulky dinitrobenzene end groups facing away from the surface, which could introduce defects in the produced SAMs.

TABLE 2
Characteristics of SAMs of ANA1, ANA2
and ANA3, given with maximum errors.

| | | Contact angle goniometry (°) | |
|---|---|---|---|
| Loading solution | Thickness of SAM (Å) | Advancing θₐ | Receeding θᵣ |
| ANA1 | 47.0 ± 0.9^{a} | 66 ± 2^{b} | 52 ± 1^{b} |
| ANA2 | 48.4 ± 0.7^{a} | 53 ± 2^{b} | 38 ± 1^{b} |
| ANA3 | 49.8 ± 0.5^{a} | 51 ± 3^{b} | 34 ± 1^{b} |

### Immobilization of ABTNT

The ability to bind ABTNT has been evaluated for the different TNT-analogues in their different mixing ratios. Several techniques have been used and the agreement between them is striking.

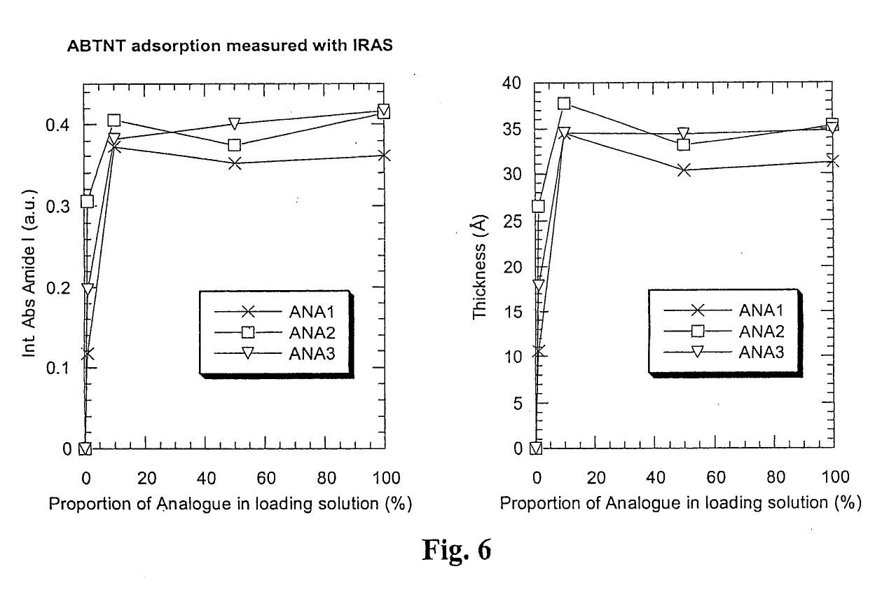

The two diagrams, illustrated in FIG. 6, are based on IRAS and ellipsometric measurements and show the amount of immobilized ABTNT for the different analogues and their mixing ratios with EG₄. For the IRAS data, part of amide I band was used as a measure of bound ABTNT, integrating between 1710-1665 cm⁻¹. The ellipsometric data shows the increase in film thickness after incubation in ABTNT. Here, EG₄ shows its protein repellent properties. The amount of ABTNT immobilized is virtually zero. Furthermore, the binding of the antibody is quite similar for the three SAMs containing high amounts of analogue. The SAMs assembled from 1% analogue solutions generally displays a lower degree of immobilization.

### Functionality Test

The functionality of the three TNT-analogues in their different mixing ratios has been evaluated with two aspects in mind. First, their ABTNT-binding capacity has been considered and second, the dissociation of ABTNT in response to TNT exposure. In both cases the two real-time techniques, SPR and QCM, have been employed. For the SPR measurements an increase in response units (RU) corresponds to an increased amount of bound ABTNT on the surface, while for QCM experiments a frequency drop is the equivalent. All three analogues possess a high potential, but focus has been on ANA1, since ii displayed a slightly better performance than the others. For all experiments in this section the running buffer was PBS (pH7.4) and both ABTNT and TNT solutions were prepared in the same medium. The ABTNT concentration was always 0.02 g/L.

### ABTNT-Binding Capacity

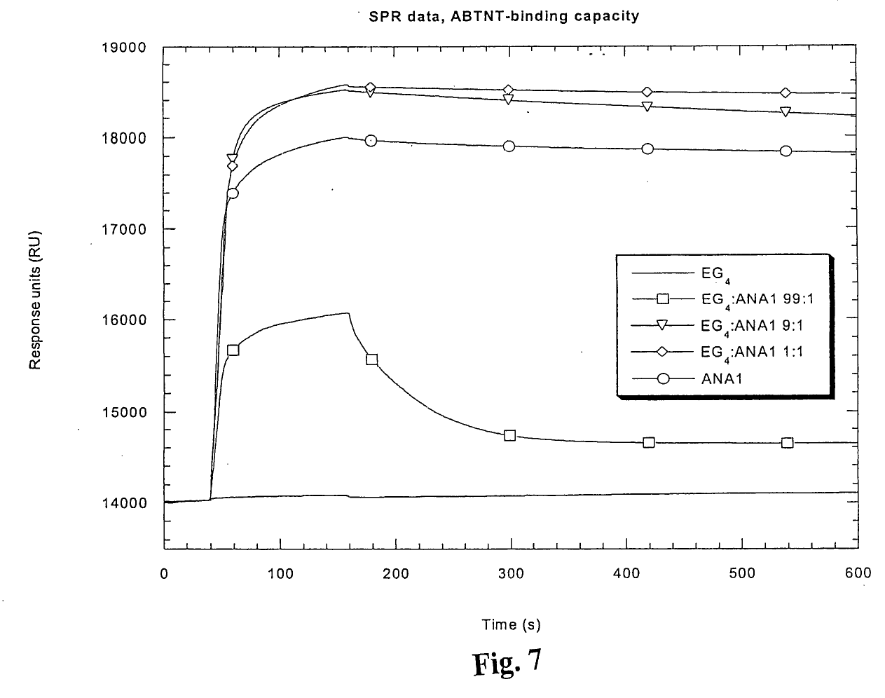

For the SPR experiments two types of instruments were used, namely a Biacore2000 and a BiacoreX system from Biacore AB. For the measurements performed on the Biacore2000 apparatus the flow rate was set to 50 μL/min and the sample surfaces were loaded by a 100 μL injection of ABTNT. In FIG. 7 the ABTNT-binding capacity of EG₄ and ANA1, and mixtures thereof, is visualized. A very low adsorption is seen for the SAM of pure EG₄ further supporting its protein repellent properties. The same mutual relationship between the different mixing ratios was found in experiments with the BiacoreX instrument run at 10 μL/min and ABTNT injection volumes of 70 μL (data not shown).

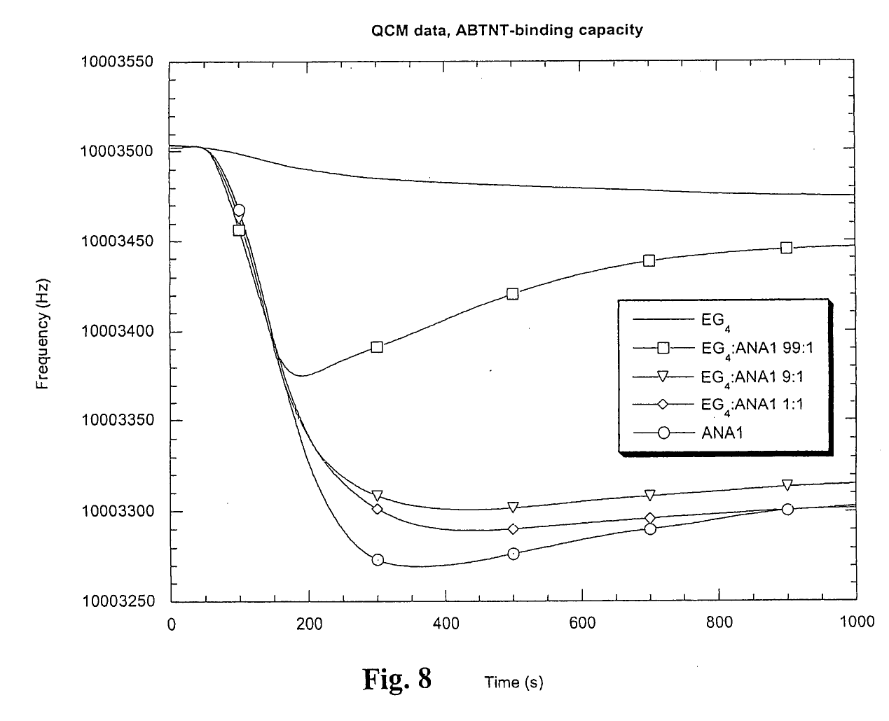

The QCM measurements were performed on a slightly modified flow cell system V2B developed by Biosensor Applications Sweden AB. All parameters were set as for the Biacore2000 experiments, i.e. flow rate 50 μL/min and ABTNT injection volume 100 L. Curves, showing the ABTNT-binding capacity of EG₄ and ANA1 and mixtures thereof, are seen in FIG. 8. Once again, the low ABTNT-binding capacity of EG₄ is clearly demonstrated.

The binding of ABTNT to the different SAMs is similar for SPR and QCM experiments. The three surfaces containing most TNT-analogue all bind ABTNT very well to the surface and the dissociation of the antibodies is very slow. A certain release of ABTNT is expected due to the constant exposure to fresh buffer, i.e. a true equilibrium can never be reached.

### ABTNT Displacement

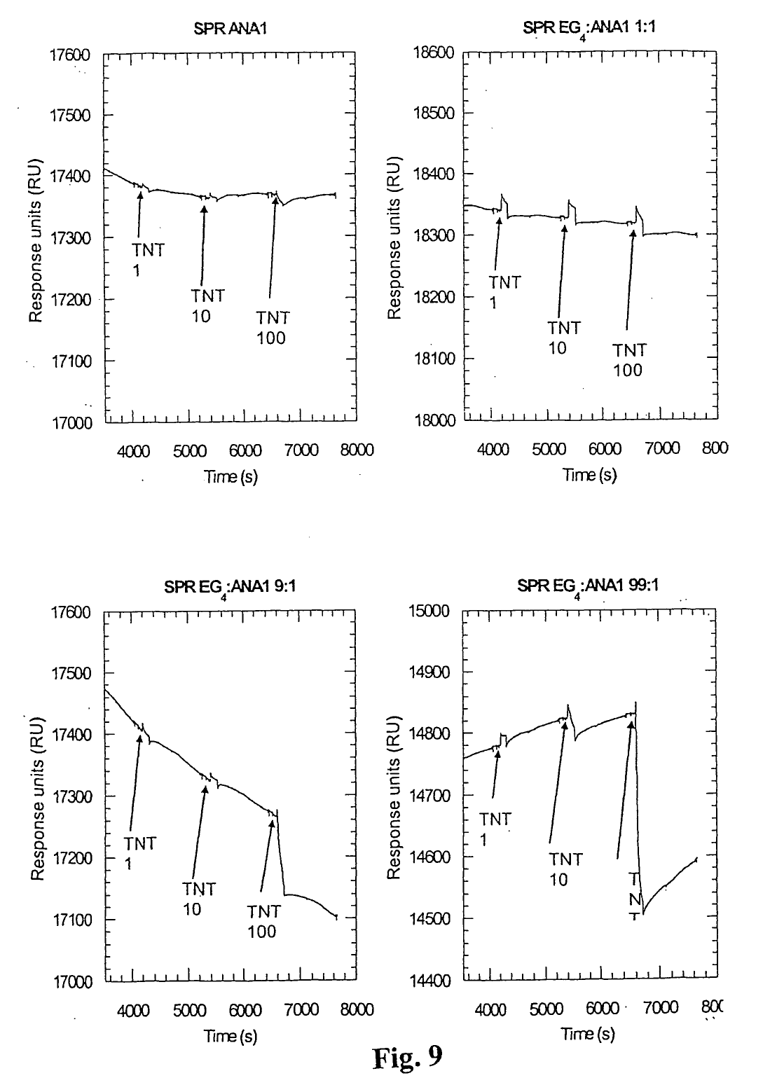

SPR curves (Biacore2000, flow rate: 50 μL/min) showing ABTNT desorption in response to TNT injection of 1, 10 and 100 pg/μL for the different mixing ratios of EG₄ and ANA1, are seen in FIG. 9. The surfaces had previously been loaded by injections of 100 μL ABTNT.

The appearance of the curves in FIG. 9 indicates that ABTNT binds weaker to the SAMs containing less ANA1, thereby facilitating the displacement reaction. This could be a consequence of the bivalency of the antibodies and their interaction with the surface. The higher the content of ANA1 is, the greater the chance for an ABTNT to find two TNT-analogues, one for each epitope, to bind to. In the case of EG₄:ANA1 99:1 this event might be less likely to occur, simply because ANA1 is less abundant. Since the binding strength of an antibody is strongly dependent on whether only one or both of its epitopes have bound to antigens, this aspect is highly relevant.

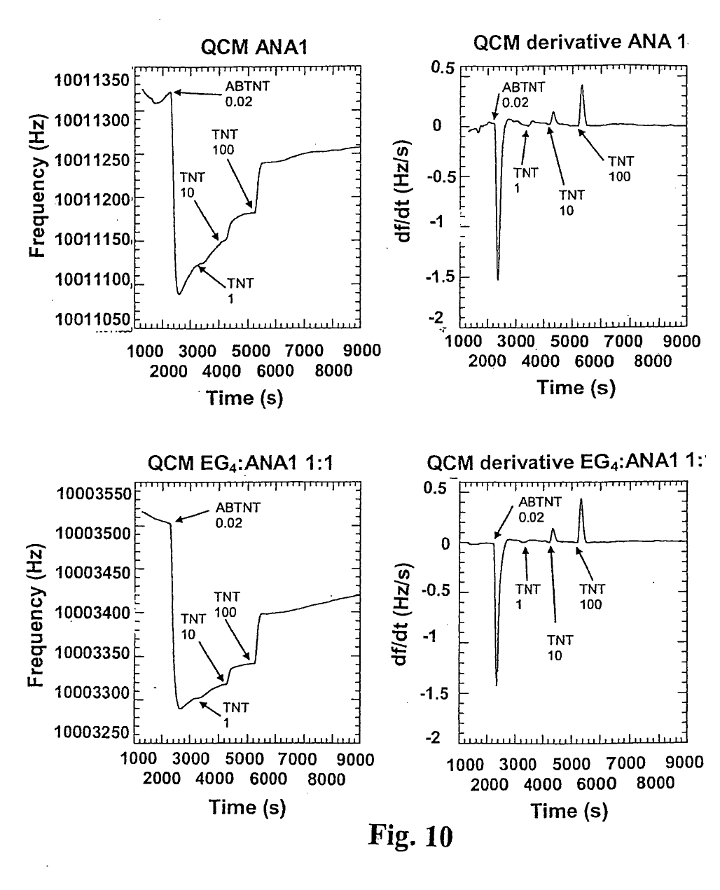

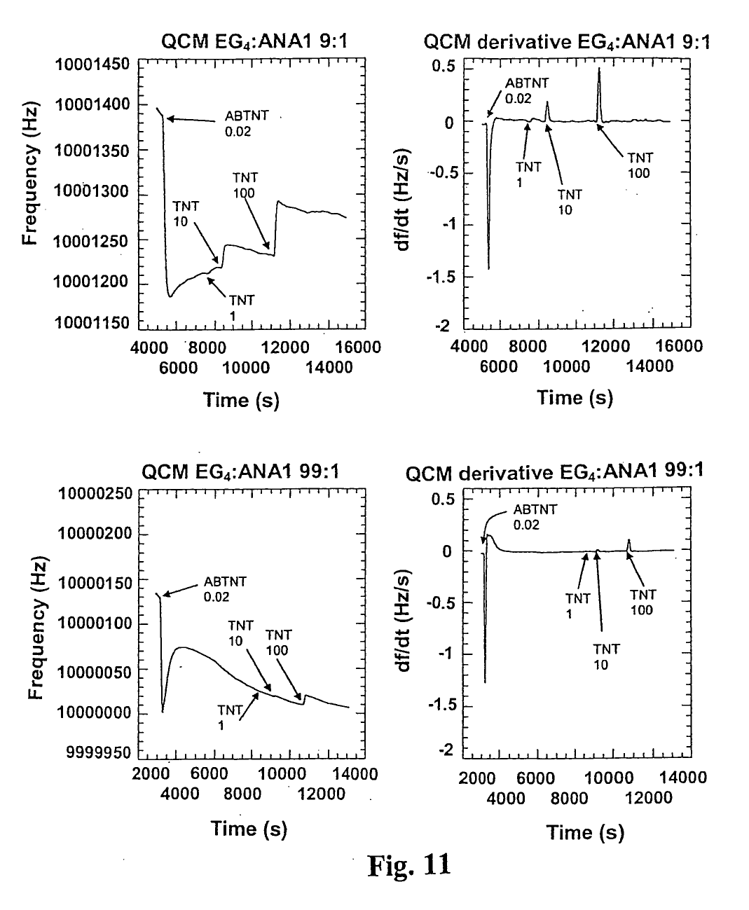

In FIGS. 10 and 11 corresponding results from QCM measurements (flow rate: 50 μL/min, injection volume: 100 μL) are shown. The mass loss caused by the TNT injections is seen as an increase in resonance frequency. The derivative of the frequency df/dt, which is proportional to the concentration, is also included, usually providing a clearer detection signal.

1. A method for preparing a mixed self-assembled monolayer (SAM) coating on a metal surface on a solid support for detection of an analyte antigen in an aqueous solution comprising,
contacting the metal surface with a mixture of i) an oligo(ethylene glycol)(OEG)-terminated amide group-containing alkyl thiols, and ii) an OEG-terminated amide group-containing alkyl thiols containing low molecular antigens bound via amide-group-formation to the OEG molecule and thereby allowing formation of the mixed self-assembled monolayer by firmly attachment via the thiol end of the groups i) and ii) to the metal surface.

2. The method of claim 1, wherein it further comprises contacting the self-assembled mixed monolayer with antibodies specific for the antigens of the SAM coating and which bond reversibly to said antigens.

3. The method of claim 1 wherein the alkyl portion in i) as well as in ii) has 1-20 methylene groups, wherein the oligo(ethylene glycol) portion in i) as well as in ii) has 1-15 ethylene oxy units.

4. The method of claim 1, wherein the metal is selected from the group consisting of gold, silver, aluminum, titanium and chromium.

5. The method of claim 1, wherein the antigen containing OEG-molecules (ii) contain different antigens which are bound in patches on the solid support.

6. The method of claim 1, wherein the antigen is selected from the group consisting of explosives and narcotics.

7. The method of claim 6, wherein the antigen is an explosive selected from the group consisting of trinitrotoluene (TNT), dinitrotoluene (DNT), hexahydro-1,3,5-trinitro-1,3,5-triazine (RDX), octahydro-1,3,5,7-tetranitro-1,3,5,7-tetrazine (HMX), pentaerythritol tetranitrate (PETN), and nitroglycerine (NG).

8. The method of claim 6, wherein the antigen is a narcotic selected from the group consisting of cocaine, heroin, amphetamine, methamphetamine, cannabinols, tetrahydrocannabinols (THC), and methylenedioxy-N-methylamphetamine (Ecstacy).

9. The method of claim 1, wherein the solid support is a piezoelectric crystal electrode or a glass plate or prism.

10. The method of claim 1, wherein the oligo(ethylene glycol) has 4-6 ethylene oxy units and the alkyl group has 15 methylene units.

11. An analysis device for detection in an aqueous solution of an analyte antigen by a displacement reaction which comprises a detector which contains as a part of the analysis device, the self-assembled monolayer prepared by the method of claim 1.

12. A method of detecting analyte antigens in an aqueous solution comprising activating, if necessary, the SAM coated metal surface on a solid support of the device of claim 11 by contacting antibodies, specific for the antigens of the SAM coating, with the SAM coated metal surface and allowing binding of the antibodies to the antigens of the SAM coating, removing excess antibodies and thereafter contacting the aqueous solution possibly containing the analyte antigens with the antibodies reversibly bound to the antigens of the coating and allowing the antibodies to dissociate and react with the analyte antigens, and thereby detecting the loss of mass on the coated metal surface by means of the analysis device.

13. A method according to claim 12, wherein the analysis device is selected from the group consisting of a Piezoelectric Quarts Crystal Microbalance device and a Surface Plasmon Resonance biosensor device.

14. The method according to claim 12, wherein the analysis device comprises a flow cell in which the SAM coated metal surface on a solid support is contained.

16. The method of claim 1, wherein the component i) of the monolayer is protein repellant.

17. A coated metal surface on a solid support prepared by the method of claim 1.
